(19) United States
(12) Patent Application Publication
(10) Pub. No.: US 2010/0274260 A1
(43) Pub. Date: Oct. 28, 2010

(54) Single Use, Disposable Uterine Manipulator and Method of Use

(75) Inventors: Francis D'Arpiany, Vendat (FR); Yves Arboy, Neuilly sur Seine (FR)

Correspondence Address: HOFFMANN & BARON, LLP, 6900 JERICHO TURNPIKE, SYOSSET, NY, 11791, US

(73) Assignee: VECTEC, Hauterive (FR)

(21) Appl. No.: 12/747,240

(22) PCT Filed: Dec. 12, 2007
(86) PCT No.: PCT/IB07/04428

§ 371 Date: Jun. 10, 2010

(51) Int. Cl. A61B 17/42

(52) U.S. Cl. 606/119

(57) ABSTRACT

The present invention relates to single use, disposable, uterine manipulator device and methods for use thereof. The device comprises a tubular elongate portion defining a longitudinal axis of the device, and having a proximal and a distal extremity, wherein the distal extremity is provided with sealing abutment means for sealingly abutting the cervix, and the distal extremity is further provided with uterine wall suction means distributed around the distal extremity for providing suction to the device, thereby enabling said device to come into suction contact with an inner uterine wall.

The present invention relates to single use, disposable surgical instruments in general, and more particularly to a device for manipulating a uterus that is particularly adapted to use in coelioscopic surgery.

One of the current problems encountered in coelioscopic surgery in female patients is the position that the female reproductive organs, and in particular the uterus, the fallopian tubes and ovaries, adopt because of the way the patient is laid out on the operating table. This position, know as the Trendelebourg position, causes the female reproductive organs to fall back into the Douglas' pouch or rectouterine excavation, thereby making access to that pouch difficult or impossible for the surgeon. Several attempts have been made to produce devices that would assist the surgeon in gaining access to the Douglas' pouch more easily, most of which involve inflicting some kind of trauma on the female reproductive organs.

For example, one known device comprises a frustoconical screw-threaded plug that is screwed into the cervix of the female patient, in order to permit manipulation of the uterus, and thereby enable the surgeon to position the uterus as desired during surgery. The problem with a device such as this is that the screw thread bites into the cervical wall, and inevitably causes a certain degree of trauma. It has been noticed in general that trauma to the cervical wall, in whatever form, can lead to reduced fertility, especially in younger female subjects, and so it would be desirable to avoid, where possible, inflicting any such trauma.

Another example of a device for uterine manipulation involves an inflatable balloon, that is provided at a distal tip of a manipulator. The manipulator is inserted into the uterus via the vagina, and the balloon inflated so that the exterior walls of the balloon come into pressure contact with the inner walls of the uterus. The idea is that the expansion of the balloon causes sufficient friction against the inner uterine wall to enable the surgeon to operate the manipulator and thereby displace the uterus in the desired direction. However, in order to be effective, the device must generally be inserted into the uterus until it meets with the upper inner wall of the latter, and then the balloon inflated, and the pressure thereby exerted by the balloon can cause localised damage to the uterine inner wall.

The device according to the present invention attempts to overcome the above limitations, whilst at the same time being easy to use and operate, and most notably, being atraumatic, i.e. not inflicting trauma on the inner wall of the uterus or the cervical wall.

Accordingly, one object of the present invention is a single use, disposable uterine manipulator device, comprising:

a tubular elongate portion defining a longitudinal axis of the device, and having a proximal and a distal extremity;

wherein the distal extremity is provided with sealing abutment means for sealingly abutting the cervix, and the distal extremity is further provided with uterine wall suction means distributed around the distal extremity for providing suction to the device, thereby enabling said device to come into suction contact with an inner uterine wall.

Preferably, the tubular elongate portion is made of a sterilisable material, preferably stainless steel, however other materials may also provide satisfaction, provided that they are suitable for use in a surgical instrument, are suitably rigid and can be sterilised.

In one preferred embodiment, the sealing abutment means and the uterine wall suction means are formed integrally as a sleeve that fits over the distal extremity of the tubular elongate portion. It is of course possible to envisage that the sealing abutment means and the uterine wall suction means be separate elements mounted on the tubular elongate portion such that they perform their function appropriately.

Preferably, the sleeve of the uterine wall suction means is made of a sterilisable polymer material, and even more preferably of a polymer material selected from the group consisting of polyoxymethacrylate, polyacetal, and PA66.

In accordance with another preferred embodiment, the tubular elongate portion is open at the distal extremity and together with an inner surface of the sleeve mentioned above defines a space allowing for the passage of fluid. The passage of fluid can be simply formed by providing for a difference in diameter between the outside diameter of the tubular elongate portion and the inner diameter of the sleeve, and leaving the distal extremity of the tubular elongate portion free or unblocked, such that fluids may pass either into the distal extremity of the elongate portion, for example, air being withdrawn under vacuum, or in the opposite direction, for example, when a liquid, such as a contrast or washing agent, is injected from the proximal end of the tubular elongate portion.

The sleeve also preferably comprises a plurality of suction orifices which traverse a wall of the sleeve and communicate with the space allowing for passage of fluid. Thes plurality of orifices is even more preferably distributed about the sleeve in a manner enabling a sufficient portion of the sleeve to come into suction contact with an inner uterine wall. Thus the plurality of orifices forms, along with the space created between the sleeve and elongate tubular portion, a way of creating fluid communication between an inner wall of the uterus, and the outside environment, i.e. the environment outside of the uterus.

Where fluid communication to be created is in the form of vacuum or suction, such suction is preferably provided at a vacuum pressure comprised between 0.4 bar and 0.8 bar, and most preferably is about 0.6 bar. The applicant has noticed that this latter value is completely atraumatic for the tissue of the uterus, including the inner walls of the uterus to which the device becomes attached when vacuum or suction is applied through the device.

Even more preferably, the device sleeve comprises a total of 8, or most preferably 10 orifices, distributed evenly about the sleeve in a 3-2-3, or respectively, a 3-2-3-2 configuration. Such a configuration allows for optimal coverage of the inner uterine wall surfaces, thereby facilitating manipulation of the uterus when vacuum is being applied.

It will be understood from the above, that the sleeve can preferably have a section that widens from a proximal end towards a distal end of said sleeve.

The sealing abutment means are designed to abut, in a sealing manner, the cervix, and thus it is preferred that they be located at a proximal end of the sleeve, and have a section that is substantially conical, adapted in shape and size to sealingly abut a cervix, and extending from a widened proximal end to a narrowed distal end which abuts or adjoins the proximal end of the sleeve. It is to be noted that since the sealing abutment means are substantially conical, the nose or distal end formed thereby can preferably be snubbed or rounded, to enable a less traumatic contact with the cervix. One should note here the complete contrast with prior art solutions involving a screw thread which engages the tissue of the cervix and can cause damage thereto.

In one advantageous embodiment, the device further preferably comprises grip means, mounted in axial sliding engagement around the tubular elongate portion, proximal to the uterine wall suction means and sealing abutment means, and distal to the proximal extremity of the tubular elongate portion. Preferably, the grip means are mounted in fixed rotating engagement about the tubular elongate portion, but they can also be made to rotate in and out of a locked position, should that be desired. It can thus be seen that the tubular elongate portion bearing the uterine wall suction means can slide along inside the grip means, while leaving an exterior grip surface free for the surgeon to manipulate the device, for example, rotating it in one direction or another.

Even more preferably, the device also comprises a Luer lock 2-way valve located at the proximal extremity the tubular elongate portion. Such a 2-way Luer lock preferable provides:

in a first open position, for a fluid communication between the elongate tubular portion and the uterine wall suction means,

in a second position, for complete obstruction of the Luer lock.

In still yet another preferred embodiment, the device further comprises hysterectomy guide means. These guide means are used to aid the surgeon in performing a hysterectomy, either a sub-total hysterectomy or a total hysterectomy, depending on how much of the cervix is removed along with the uterus. Preferably, the hysterectomy guide means comprises an adapter comprising engagement means to engage the proximal end of the sealing abutment means, and ablation guide sleeve which is adapted to engage at least partially circumferentially an exterior wall of the cervix. Even more preferably, the adapter has clips means for removably engaging a proximal surface of the sealing abutment means. It can thus be seen that the adapter slides over the sealing abutment means and can be clipped and retained at a proximal end of the sealing abutment means. Where the adapter is made of resilient or slightly elastic material, it is preferably designed to elastically fit over the substantially conical sealing abutment means and then snap back over a proximal circumference or periphery of said sealing abutment means.

As a result, it is preferred that the adapter also be substantially conical in shape, extending from a widened proximal end towards a narrowed distal end, the shape defined thereby enabling sealing abutment of the adapter with an inner vaginal wall. Thus, the initial cervical sealing abutment means no longer serve their purpose, since they are covered, in this particular embodiment, by the adapter, which is dimensioned to provide sealing abutment with an inner vaginal wall and not the cervix.

In a further preferred embodiment, the ablation guide sleeve is substantially tubular, in order to be able to be slid over the device as described initially, but also comprises a peripheral projecting wall, which extends from a proximal end in abutment with the distal end of the adapter, towards a distal end, wherein the projecting wall engages at partially circumferentially an exterior wall of the cervix. In this way, the projecting wall serves as a guide for the surgeon as to where the tissue of the cervix should be sectioned and removed along with the rest of the uterus.

In still yet another object of the invention, there is provided a method of manipulating an inner wall of a uterus, comprising the steps consisting of:

inserting a uterine manipulator device comprising a tubular elongate portion defining a longitudinal axis of the device, and having a proximal and a distal extremity, wherein the distal extremity is provided with sealing abutment means for sealingly abutting the cervix, and the distal extremity is further provided with uterine wall suction means distributed around the distal extremity for providing suction to the device;

applying suction vacuum pressure to the device to activate suction via the uterine wall suction means, thereby enabling said device to come into suction contact with an inner uterine wall;

moving the device into a desired position, whereby the suction applied via the uterine wall suction means causes the uterus to be moved correspondingly with the device.

The invention also provides for a method for performing a contrast diagnostic test in a female reproductive organ, comprising the steps consisting of:

introducing a uterine manipulation device as described above into a uterus to obtain sealing abutment of the device with a cervix;

setting a Luer lock on the uterine manipulator device to a position whereby vacuum suction communication is obstructed and yet still allowing another fluid to be introduced into said device; and

introducing via said Luer lock a contrast agent that flows through the device and into the uterus.

In addition, still yet another preferred object is a method for performing a hysterectomy, comprising the steps consisting of:

introducing a device as described above into a uterus, whereby an adapter comes into sealing abutment with an inner vaginal wall, and a ablation guide sleeve comes into at least a partial circumferential engagement with an exterior wall of a cervix;

using a distal extremity of the ablation guide sleeve as a guide to determine the limit of ablation of said uterus.

Other objects and preferred embodiments will become apparent through the detailed description and the figures, given purely for illustrative purposes, as follows :

### BRIEF DESCRIPTION OF THE FIGURES

FIG. 1 is schematic represenation of a single use, disposable, uterine manipulation device according to the present invention;

FIGS. 2a, 2b and 2c are different schematic views of a distal part of the uterine manipulation device illustrated in FIG. 1;

FIG. 3 is a first representation of the introduction into a uterus of the device of FIG. 1;

FIG. 4 is a second representation of the normal final position of the device of FIG 1 once fully introduced into the uterus;

FIGS. 5a, 5b and 5c are different schematic representations of a further adapter for the device of the present invention;

FIG. 6 is a schematic representation of an extra component that fits onto the adapter illustrated in FIG. 5, and which is suited to assisting a surgeon in carrying out a hysterectomy.

### EXAMPLES, OR DETAILED DESCRIPTION OF THE INVENTION

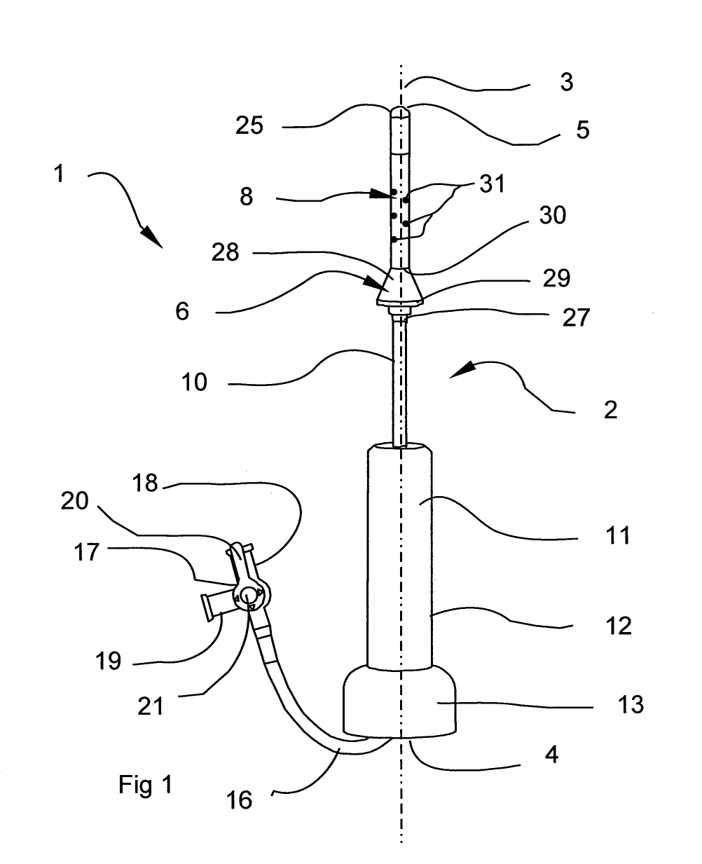

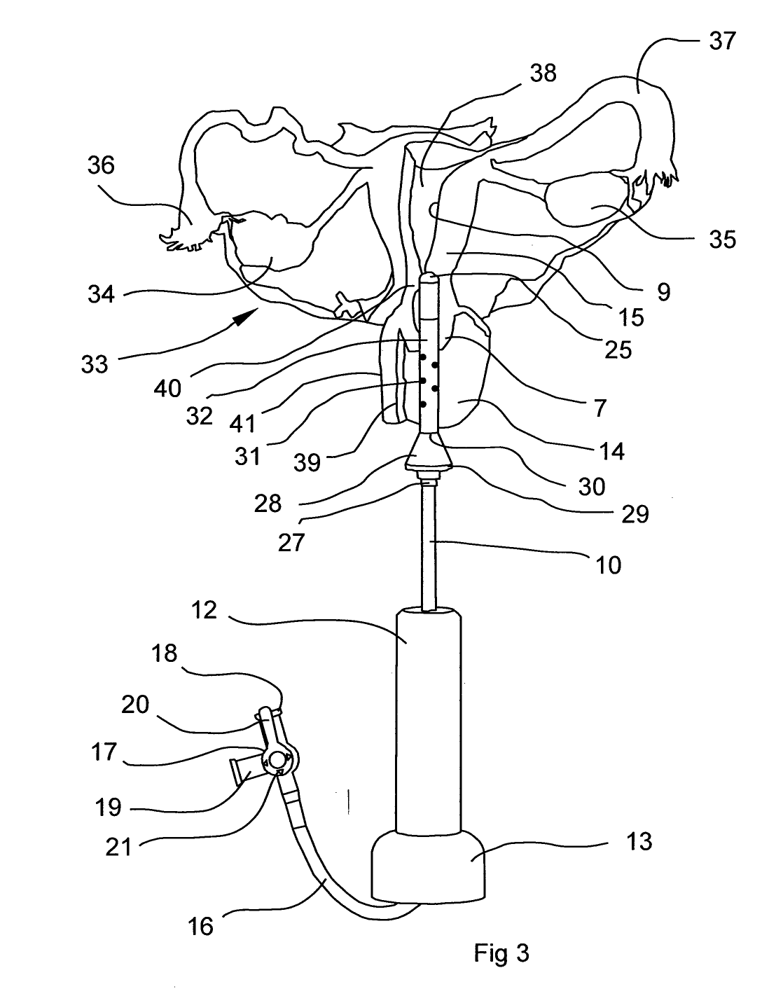

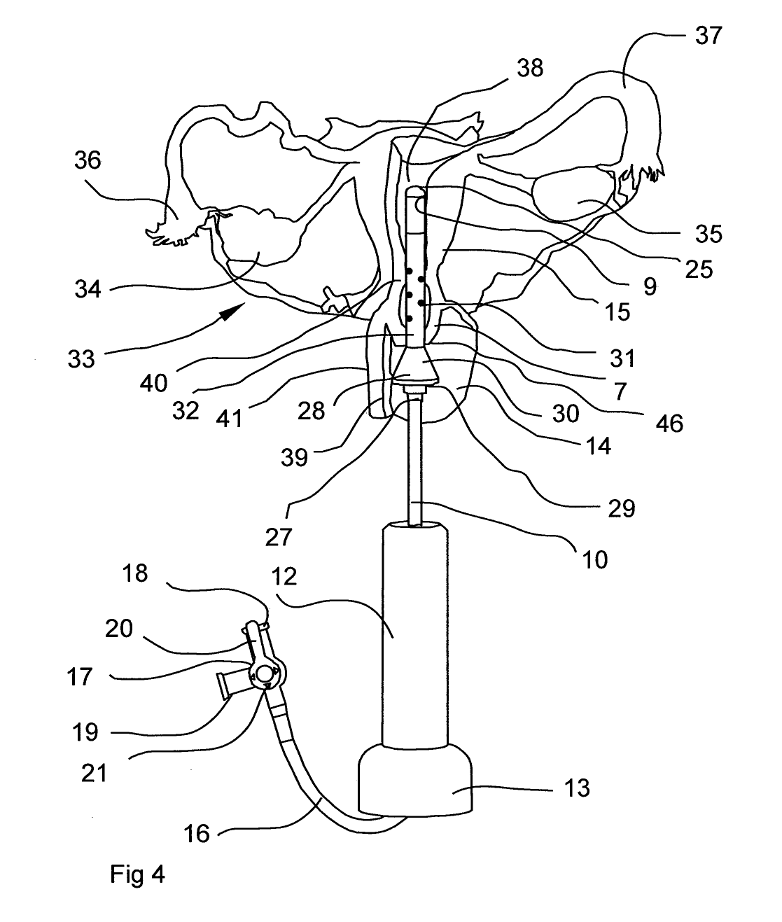

As can be seen from FIG. 1, the single use, disposable, uterine manipulation device, indicated by the general reference 1, comprises a tubular elongate portion, identified by the general reference 2, defining a longitudinal axis 3 of the device 1, and having a proximal 4 and a distal extremity 5. The distal extremity 5 is provided with sealing abutment means, indicated generally by the reference 6, for sealingly abutting a cervix 7 (cf. FIGS. 3 and 4), and the distal extremity 5 is further provided with uterine wall suction means 8 distributed around the distal extremity 5 for providing suction to the device 1, thereby enabling the device 1 to come into suction contact with an inner uterine wall 9. The tubular elongate portion 2 is made of a sterilisable, surgically appropriate material, preferably stainless steel, and thus generally takes the shape of a stainless steel tube 10. The tube 10 is essentially hollow along the axis 3 and has an opening at both the proximal 4 and distal 5 extremities. FIG. 1 also shows that the device 1 further comprises grip means, indicated generally by the reference 11, mounted in axial sliding engagement around the tubular elongate portion 2, proximal to the uterine wall suction means 8 and sealing abutment means 6 and distal to the proximal extremity 4 of the tubular elongate portion 2. The grip means 11 are mounted in fixed rotating engagement about the tubular elongate portion 2. As can be seen from FIG. 1, the grip means II comprise a fairly large outer diameter tube 12. The tube can optionally be provided with ridges, not illustrated in the figure, to facilitate prehension by the operator of the device 1. The grip means 11 further include an enlarged, approximately funnel-shaped, proximal extremity 13, enabling other instruments to be introduced into the tube 12 if necessary. Since the grip means 11 are rotatively, but not slidingly locked, about the axis 3 of the device, it is posssible to manipulate the device by rotating the tube 12 about said axis. It is furthermore possible to slide the sealing abutment means 6 and uterine wall suction means 8 along the axis 3 to introduce the latter into, and out of, the vagina 14 towards the uterus 15. The proximal extremity 4 of the device 1 is connected to a fluid communication conduit 16. The conduit 16 is made of a flexible fluid impermeable polymer material, of the type commonly used in hospitals or other surgical devices for the passage of fluids, be they liquids or gases. The fluid communication conduit 16 is connected to a Luer lock valve 17, having two ports 18,19. A first port 18 is connected to a vacuum generator device or apparatus (not shown), for establishing vacuum or suction, such that air is sucked in via the uterine wall suction means 8, through the distal extremity 5 of the device 1, and then along the tubular elongate portion 2 and through the conduit 16 and first port 18. A second port 19 is provided for introduction and removal of another fluid, for example, a contrast agent such as methylene blue, which can be injected as a liquid through the second port 19 and into the uterus 15 via the device 1 by following the same schema as above, but in a reverse direction. The 2-way Luer lock valve 17 thus provides, in a first open position, for a fluid communication between the elongate tubular portion 2 and the uterine wall suction means 8, and in a second position, for complete obstruction of the Luer lock 17. The 2 Luer lock valve 17 thus also comprises a switch or lever 20, which can be activated to set the lock in a desired position, depending on whether the operator of the device 1 wants the fluid communication to be vacuum or suction in the first case, fluid communication for the introduction of a contrast agent, in a second case, and of course a third position, whereby all fluid communication is obstructed. In order to facilitate recognition of the position in which the lock 17 is set, the lock can be provided with markings 21, arranged on the lever 20, and indexed therewith.

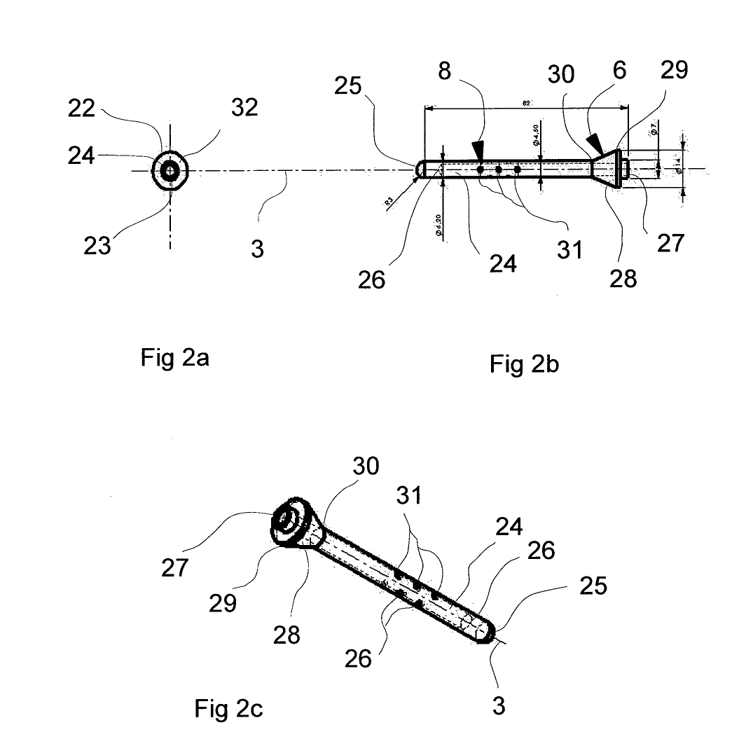

As can be seen in more detail from FIG. 2, the sealing abutment means 6 and the uterine wall suction means 8 are formed integrally as a sleeve 32 that fits over the distal extremity 5 of the tubular elongate portion 2. FIG. 2a shows a cross-section of the sleeve 32, along axis 3 of the device, clearly showing that the sleeve is hollow, and has an outer wall 22 and inner wall 23, defining an inner bore or space 24. The distal end 25 of the sleeve is blind, i.e. closed, and the distal limit 26 of the bore or space 24 has a generally concave shape, such that a distal end of the stainless steel tube 10 does not sealingly abut said limit 26, thereby allowing for passage of fluid between an opening in the distal end of the tube JO, and the space 24 of the sleeve. Additionally, as can be seen from FIG. 2b, the sleeve does not have a circular cross-section, but extends from proximal end 27 towards the distal end 25 of the sleeve in a slightly widening cross-section, thereby ensuring that a fluid communication space 24 will be created between the stainless steel tube 10 and the inner wall 23 of the sleeve 32, and that the tube 10 will be held in elastic sealing engagement with the proximal end 27. The sealing abutment means 6 are located at a proximal end 27 of the sleeve 32, and has a cross-section susbtantially forming a cone 28, adapted in shape and size to sealingly abut a cervix. The cone 28 extends from a widened proximal end 29 to a narrowed distal end 30 which abuts or adjoins the sleeve 32. The uterine wall suction means 8 also comprise a plurality of orifices 31, distributed evenly around the sleeve 32, preferably in a 3-2-3-2 configuration or arrangement, as can be seen partially from FIG. 2c. The orifices 31 traverse the outer and inner walls 22, 23 of the sleeve, and open into the fluid communication space 24. Thus, when vacuum is applied via the Luer lock port 18, air is withdrawn from within the device and a depression created that causes a uterine wall 9 to be sucked onto the sleeve 32. In a converse manner, when fluid, such as a contrast agent, is introduced via port 19, said fluid can pass through the device, into the space 24 and through the orifices into the uterus 15. As the cone sealingly abuts the cervix 7 of the uterus 15, no leakage in the sealtight environment can occur, and thus the vacuum pressure is maintained, or respectively, the fluid introduced into the uterus 15 remains therein or will permeate through the tissues therein or other organs connected thereto.

Turning now to FIG. 3, one can see a schematic representation of female human reproductive organs, indicated by the general reference 33. These organs are represented generally by the ovaries 34, 35, the fallopian tubes 36, 37, the uterine cavity 38, the cervix 7, the uterine isthmus 40, and the vagina 14. The vagina 14 and uterus are shown here o the right hand side a in partial cut-away representation, in order to show the uterine cavity 38 and inner 39 and outer vaginal 41 walls and inner uterine 9 walls. In FIG. 3, one can see that the device has been introduced via the vagina 14 and the cervix into the uterus 14, such that the distal end 25 of the sleeve 32 has reached the uterine isthmus 40. In this position, the device 1 of the invention is not yet ready for use.

FIG. 4 shows, in a schematic representation, the position of the device 1 of the invention, once introduction thereof into the uterus 15 has been completed. In this figure, it can be seen that the distal end 30 of the cone 28 of the sealing abutment means has come into sealing abutment engagement with the cervix 7, forming a substantially leaktight seal. One will also note that the sleeve 32 has now been pushed past the uterine isthmus 40, such that the distal end 25 of the sleeve 32 is located in the uterine cavity 38. The plurality of orifices 31 of the suction means 8 are now in physical contact with an inner uterine wall 9. In FIG. 4, the Luer lock port 18 is shown in the closed position, but when the lever 20 is moved and set to the open position, i.e. in this particular example by turning the lever through 90 degrees so that it lies over the second port 19, then vacuum pressure that can be applied will suck out any air in the device, creating a depression in the space 24 between the inner wall of the sleeve 32 and stainless steel tube and thereby causing a drop in pressure at the orifices 31, which in turn will cause the inner uterine wall 9 to be sucked onto the outer wall 22 of the sleeve 32. It thus becomes possible to manipulate the uterus 15 and associated organs and put them into a position more suitable for the surgeon, simply by moving the device around in the three-dimensional anatomical space. In this way, it also makes the Douglas' pouch accessible to the surgeon, should that be required. The seal is maintained by the conical shape of the cone 28 which abuts sealingly against the cervix 7, independently of the angle of inclination of the device. In a similar maner, a diagnostic test, for example, the methylene blue test, can be carried out very simply by activating the lever 20 again, to set the Luer lock port 18 into the closed position, for example, by either moving the lever 20 forward again by 90 degrees, or by reversing the initial movement. This movement will then open port 19, through which the methylen blue contrast agent can be injected. As there is a leaktight seal against the cervix 7, none of the fluid introduced can escape and will therefore diffuse in a classical manner through the various tissues, and particularly up the fallopian tubes to the ovaries, where the contrast agent will become visible through a coelioscope.

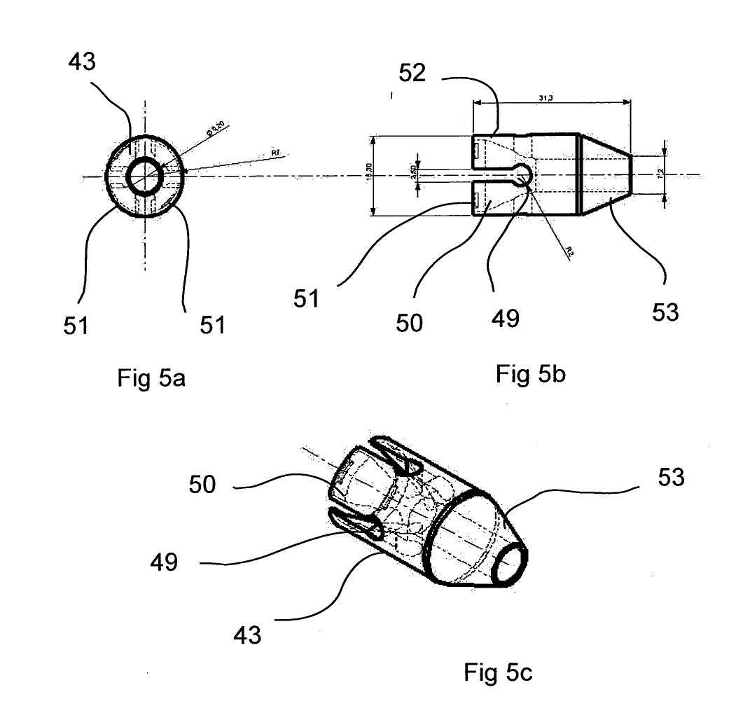

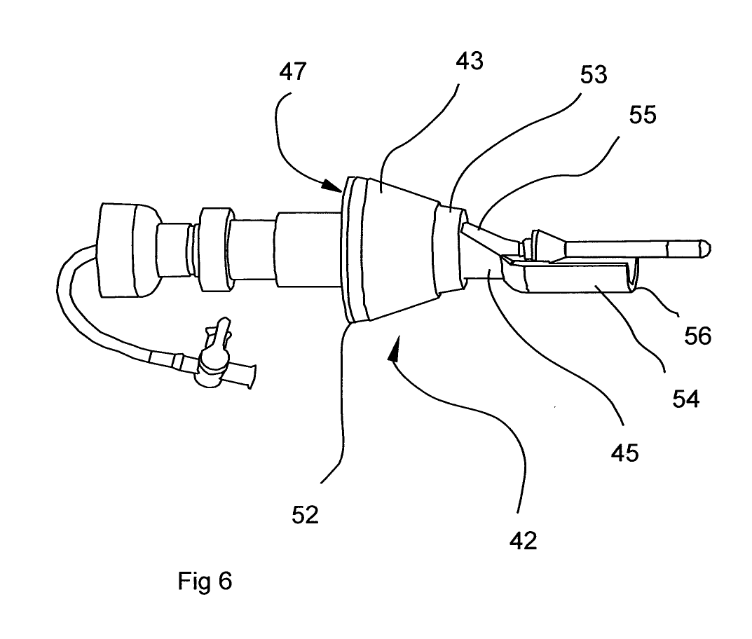

Turning now to FIGS. 5 and 6, these are schematic representations of a further embodiment of the device of the present invention, in which said uterine manipulator device 1 can be adapted to guide the surgeon who needs to carry out a hysterectomy, be it a total or sub-total hysterectomy. Consequently, the device 1 further comprises hysterectomy guide means, indicated by the general reference 42 comprises an adapter 43 comprising engagement means 44 to engage the proximal end 27 of the sealing abutment means 6, and ablation guide sleeve 45 which is adapted to engage at least partially circumferentially an exterior wall 46 of the cervix 7. The adapter 43 preferably has clip means 47 for removably engaging a proximal surface 48 of the sealing abutment means 6. In FIG. 5, it can be seen that the adapter 43 has lands or cut-outs 49, which form wings 50 in the adapter 43 and enable it to be slid over the cone 28 via the distal end of the sealing abutment means 6. Since the adapter is preferably made of resilient or elastic material, the wings 50 spread as the adapter 43 is slid over the cone 28 towards the widened proximal end 29 of the latter. The clip means 47, which can for example be one or more radial flanges 51 provided on the wings 50, then reach over the widened proximal end 29 of the cone and clip into place on a contact surface 48 which is essentially the proximal surface of the widened proximal end 29. Such a clipping action provides for sufficient maintainance of the adapter 43 in position, whilst at the same time allowing for its release simply by easing a radial flange back over onto the cone 28. Consequently, the adapter 43 is preferably substantially conical in shape, extending from a widened proximal end 52 towards a narrowed distal end 53, the shape defined thereby enabling sealing abutment of the adapter 43 with an inner vaginal wall 39. The ablation guide sleeve 45 also comprises a peripheral projecting wall 54, which extends from a proximal end 55 in abutment with the distal end 53 of the adapter 43, towards a distal end 56, wherein the projecting wall 54 engages partially circumferentially an exterior wall of the cervix 46. In this way, the projecting wall 54 serves to guide the surgeon wishing to remove the uterus 15 down to a limit on the cervix 7 that is defined by the extent of the projecting peripheral wall 54.

1. A single use, disposable, uterine manipulator device, comprising:
a tubular elongate portion defining a longitudinal axis of the device, and having a proximal and a distal extremity;
wherein the distal extremity is provided with sealing abutment means for sealingly abutting a cervix, and the distal extremity is further provided with uterine wall suction means distributed around the distal extremity for providing suction to the device, thereby enabling said device to come into suction contact with an inner uterine wall.

2. A uterine manipulator device according to claim 1, wherein the tubular elongate portion is made of a sterilisable material.

3. A uterine manipulator device according to claim 1, wherein the sealing abutment means and the uterine wall suction means are formed integrally as a sleeve that fits over the distal extremity of the tubular elongate portion.

4. A uterine manipulator device according to claim 3, wherein the sleeve is made of a sterilisable polymer material, selected from the group consisting of polyoxymethacrylate, polyacetal, and PA66.

5. A uterine manipulator device according to claim 1, wherein the tubular elongate portion is open at the distal extremity and together with an inner surface of the sleeve defines a space allowing for the passage of fluid.

6. A uterine manipulator device according to claim 1, wherein the sleeve comprises a plurality of suction orifices which traverse a wall of the sleeve and communicate with the space allowing for passage of fluid.

7. A uterine manipulator device according to claim 6, wherein the plurality of orifices is distributed about the sleeve in a manner enabling a sufficient portion of the sleeve to come into suction contact with an inner uterine wall.

8. A uterine manipulator device according to claim 1, wherein the suction is provided at a vacuum pressure between 0.4 bar and 0.8 bar.

9. A uterine manipulator device according to claim 1, wherein the sleeve comprises a total of 10 orifices, distributed evenly about the sleeve in a 3-2-3-2 configuration.

10. A uterine manipulator device according to claim 1, wherein the sleeve has a cross-section that widens from a proximal end towards a distal end of said sleeve.

11. A uterine manipulator device according to claim 1, wherein the sealing abutment means are located at a proximal end of the sleeve, and has a section that is substantially conical, adapted in shape and size to sealingly abut a cervix, and extending from a widened proximal end to a narrowed distal end which abuts or adjoins the sleeve.

12. A uterine manipulator device according to claim 1, wherein the device further comprises grip means, mounted in axial sliding engagement around the tubular elongate portion, proximal to the uterine wall suction means and sealing abutment means and distal to the proximal extremity of the tubular elongate portion.

13. A uterine manipulator device according to claim 1, wherein the grip means are mounted in fixed rotating engagement about the tubular elongate portion.

14. A uterine manipulator device according to claim 1, wherein the device further comprises a Luer lock 2-way valve located at the proximal extremity the tubular elongate portion.

15. A uterine manipulator device according to claim 14, wherein the 2-way Luer lock provides:
in a first open position, for a fluid communication between the elongate tubular portion and the uterine wall suction means, and
in a second position, for complete obstruction of the Luer lock.

16. A uterine manipulator device according to claim 1, wherein the device further comprises hysterectomy guide means.

17. A uterine manipulation device according to claim 16, wherein the hysterectomy guide means comprises an adapter comprising engagement means to engage the proximal end of the sealing abutment means, and an ablation guide sleeve which is adapted to engage at least partially circumferentially an exterior wall of the cervix.

18. A uterine manipulation device according to claim 17, wherein the adapter has clips means for removably engaging a proximal surface of the sealing abutment means.

19. A uterine manipulation device according to claim 18, wherein the adapter is substantially conical in shape, extending from a widened proximal end towards a narrowed distal end, the shape defined thereby enabling sealing abutment of the adapter with an inner vaginal wall.

20. A uterine manipulation device according to claim 16, wherein the ablation guide sleeve comprises a peripheral projecting wall, which extends from a proximal end in abutment with the distal end of the adapter, towards a distal end, wherein the projecting wall engages partially circumferentially an exterior wall of the cervix.

21. A method of manipulating an inner wall of a uterus, comprising the steps of:
inserting a uterine manipulator device comprising a tubular elongate portion defining a longitudinal axis of the device, and having a proximal and a distal extremity, wherein the distal extremity is provided with sealing abutment means for sealingly abutting the cervix, and the distal extremity is further provided with uterine wall suction means distributed around the distal extremity for providing suction to the device;
applying suction vacuum pressure to the device to activate suction via the uterine wall suction means, thereby enabling said device to come into suction contact with an inner uterine wall; and
moving the device into a desired position, whereby the suction applied via the uterine wall suction means causes the uterus to be moved correspondingly with the device.

22. A method for performing a contrast diagnostic test in a female reproductive organ, comprising the steps of:
introducing a uterine manipulation device according to claim 1 into a uterus to obtain sealing abutment of the device with a cervix;
setting a 2-way Luer lock on the uterine manipulator device to a position whereby vacuum suction communication is obstructed and yet still allowing another fluid to be introduced into said device; and
introducing via said Luer lock a contrast agent that flows through the device and into the uterus.

23. A method for performing a hysterectomy, comprising the steps of:
introducing a device according to claim 1 into a uterus, whereby an adapter comes into sealing abutment with an inner vaginal wall, and an ablation guide sleeve comes into at least a partial circumferential engagement with an exterior wall of a cervix; and
using a distal extremity of the ablation guide sleeve as a guide to determine the limit of ablation of said uterus.
